(19) United States
(12) Patent Application Publication
(10) Pub. No.: US 2011/0071482 A1
(43) Pub. Date: Mar. 24, 2011

(54) DEVICES AND METHODS FOR SIGNALING WHEN ACTION IS DUE IN RELATION TO A MEDICAL DEVICE

(76) Inventor: James R. Selevan, Laguna Beach, CA (US)

(21) Appl. No.: 12/888,352

(22) Filed: Sep. 22, 2010

Related U.S. Application Data
(63) Continuation-in-part of application No. 12/322,010, filed on Jan. 28, 2009.
(60) Provisional application No. 61/277,210, filed on Sep. 22, 2009.
(60) Provisional application No. 61/248,815, filed on Oct. 5, 2009.
(60) Provisional application No. 61/397,128, filed on Jun. 7, 2010.

(51) Int. Cl. A61M 35/00; G08B 19/00; G08B 1/08

(52) U.S. Cl. 604/307; 604/304; 340/522; 340/539.12

(57) ABSTRACT

Methods and devices for signaling to a subject or subject's care giver the occurrence of at least one event selected from: a) expiration or impending expiration of a predetermined time period and/or b) a change in a physiological or device-associated variable, associated with a medical device that is positioned on, in or connected to the subject's body.

### RELATED APPLICATIONS

This application is a continuation in part of copending U.S. patent application Ser. No. 12/322,010 filed Jan. 28, 2009. Also, this application claims priority to U.S. Provisional Patent Applications No. 61/277,210 filed Sep. 22, 2009; 61/248,815 filed Oct. 5, 2009; 61/225,434 filed Oct. 27, 2009 and 61/397,128 filed Jun. 7, 2010. The entire disclosure of each of the foregoing patent applications is expressly incorporated herein by reference.

### FIELD OF THE INVENTION

The present invention relates generally to medical devices and methods and more particularly to apparatus and methods for signaling when some action (e.g., changing, removal, adjustment, replacement, replenishment, etc.) is due in relation to a medical catheter, transcutaneous substance delivery device or other medical device positioned on or in the body of a human or animal subject.

### BACKGROUND

Pursuant to 37 CFR 1.71(e), this patent document contains material which is subject to copyright protection. The copyright owner has no objection to facsimile reproduction of the entire patent document or the patent disclosure, as it appears in the Patent and Trademark Office patent file or records, but otherwise reserves all copyright rights whatsoever.

Various catheters and other medical devices are commonly inserted or otherwise positioned such that all or part of the catheter or other device dwells within a subject's body for a period of time. In many cases, it is desirable for some action (e.g., changing, removal, adjustment, replacement, replenishment, etc.) to be taken in connection with such devices at a predetermined time or upon occurrence of a predetermined change in a variable. For example, it is typically desirable to remove or replace certain indwelling catheters and medical devices after they have been within the subject's body for a specified time period. Also, it is typically desirable to remove or replace certain indwelling catheters and medical devices upon occurrence of a change in a variable (e.g., elevation of local skin or body temperature) indicative of some complication or untoward effect (e.g., microbial infection).

In particular, intravenous, intra-arterial, intravesicular (bladder), intracranial or other invasive conduits (e.g., catheters) are commonly used in inpatient as well as some outpatient settings and can be prone to inflammation or infection. The likelihood of infection and/or inflammation is time-dependent. The longer the foreign tubing is situated within the body the greater the likelihood of infection. A local infection at the site of insertion may be contained, or the infection may spread from the local site to produce a systemic sepsis. These latter conditions may be life-threatening. Even in a non-life-threatening local infection, the presence of infection may delay hospital discharge. This delay often is quite expensive. Therefore, any device that helps to prevent local infection at the site of an indwelling catheter or tube can improve quality and lower costs

Many hospitals or outpatient facilities incorporate a written policy and procedure manual that defines the proper interval for changing the site of insertion of tubing/catheters. Also, manufacturers of these indwelling devices typically call for removal at an interval of 3 or 4 days. However, for a variety of reasons this duration may be exceeded. The purpose of this invention is to remind (notify) clinical personnel that the time to change the catheter or tubing has arrived. Failure to change such tubing subjects the patient to greater risk. The visual or audible notification is also available to the patient and family, thereby assuring greater scrutiny over the status of the indwelling conduit.

Should an infection develop at the site of an indwelling catheter, one would expect clinical signs consistent with such a development. These signs include redness, swelling, pain, and heat. This invention is able to detect changes in temperature of the skin early in the development of an infection and notify clinical personnel using a visual, vibration, or audible alert. In addition, the patient and their family are now considered the “last line of defense” against medical error. Through the use of a visual, vibration, or audible alert the patient or caregiver can be made aware of an impending clinical complication. By positioning or incorporating the signaling system (e.g., components comprising the sensing element, power source, microcontroller and support circuitry) directly within or on the catheter or medical device itself or within/on an insertion needle, sheath, infusion or drainage tubing, “securement” device (for example, StatLock® stabilization device, C.R. Bard, Inc., Murray Hill, N.J.), connector, or other apparatus that is routinely attached to or associated with the catheter or other inserted medical device, the caregiver need not take additional steps to attach or connect the signaling system of the present invention to the patient.

Should an infection or inflammation develop, one of the earliest signs is a rise in temperature of the skin in the area of the site. Core body temperature will not rise until the infection becomes systemic, that is, enters the blood stream and leads to a generalized febrile response. If the local infection can be identified prior to systemic spread, the severity and cost of treatment can be mitigated. Measuring skin temperature and correlating to core body temperature is fraught with error, and by its nature slow in identifying early infection. The unique aspect of this device is that it monitors the local site for a change in temperature, that is, delta-temperature at the site of insertion. This is accomplished not by comparing with ambient or core temperature, but by integrating the area-under-the-curve of temperature versus time, thereby following the history of temperature at that site.

There are situations where the patient must be notified of the time elapsed from a particular procedure. For example, when treating bladder cancer, medication is infused into the bladder to be retained for 1 hour. Should the patient void the medication sooner, efficacy is compromised. If the medication is retained beyond the hour, toxicity may ensue. In this clinical setting, a thin, plastic bracelet as typically worn by hospital patients incorporating the timing and alert device can ensure that the proper protocol is followed.

Also, transcutaneous delivery systems have been used to deliver various types of substances to human or animal subjects. Known types of transcutaneous delivery systems include transdermal drug delivery patches as well as wearable injectors which pump or infuse drug through one or more needles inserted through the skin.

Transdermal drug delivery patches have included single-layer drug-in-adhesive patches, multi-layer drug-in-adhesive patches, reservoir patches, matrix patches and vapor patches.

In the single-layer drug-in-adhesive type patches, the drug is contained in an adhesive layer that performs the dual functions of adhering the system to the subject's skin and releasing or eluting the drug.

In the multi-layer drug-in adhesive type patches, there are multiple drug-containing adhesive layers. One of the layers immediately releases an initial dose of the drug and other layer(s) is/are for sustained release of the drug over a subsequent time period. In reservoir systems, the drug is contained in and released from a reservoir (e.g., a cavity or bladder) and a separate adhesive layer or area is used to adhere the system to the subject's skin.

In matrix type patches, a solution or suspension of the drug is contained within a semisolid matrix and a separate adhesive layer or area is used to adhere the system to the subject's skin.

In vapor patches, various layers of the device are constructed to release vapors of essential oils or other vapors for a sustained period of time (e.g., up to 6 hours).

In many transcutaneous drug delivery systems, it is desirable for the system to remain in place on the subject's body for a sufficient time to deliver the intended therapeutic dose of the drug. Premature removal can result in sub-optimal or insufficient delivery of the drug. Also, in many cases, it is important to remove and/or replace the transcutaneous drug delivery system at a prescribed time or upon occurrence of some untoward complication that would adversely affect the rate or amount of drug delivered. Although the subject may be instructed to remove the patch on a particular day, subjects may sometimes forget to do so. Moreover, to instruct removal at a particular hour is difficult and subject to significant compliance issues.

### SUMMARY OF THE INVENTION

The present invention provides apparatus and methods for signaling when some action (e.g., changing, removal, adjustment, replacement, replenishment, etc.) is due in relation to a medical catheter or other indwelling medical device.

In accordance with one aspect of present invention, there is provided a signaling system that is associated with a catheter or other indwelling medical device, such signaling system comprising (a) a signaling apparatus and (b) a signal triggering apparatus which causes the signaling apparatus to emit a signal upon occurrence of at least one event selected from: a) expiration or impending expiration of a predetermined time period and b) a change in a physiological or device-associated variable. The signaling apparatus may comprise any suitable audible, visual or tactile signaling apparatus including but not limited to light emitting apparatus, light emitting diodes, sound emitting apparatus, tactile signaling apparatus, vibrators, shakers, etc. The signal triggering apparatus may comprise any suitable apparatus for triggering the signaling apparatus at a desired time point or upon sensing or calculation of the occurrence of some device-associated or subject-associated event, including but not limited to; a microcontroller, a chip, a timer, a clock, a semiconductor, a sensor, a temperature sensor, a galvanometer, a flow detector, an air detector, a read-only memory (ROM) device, a programmable read-only memory (PROM) device, a field programmable read-only memory (FPROM) device and a one-time programmable non-volatile memory (OTP NVM) device.

In accordance with another aspect of the present invention, the signaling system of the foregoing character may be positioned directly on or in the catheter or medical device (typically on a portion of the catheter or medical device that remains exteriorized when the catheter or other medical device is inserted or positioned in the subject's body). The types of catheters or medical devices in or on which the signaling system may be positioned include but are not limited to: catheters, intravenous catheters; intra-arterial catheters; intracranial catheters; intra-vesicular catheters, urinary catheters; Foley catheters; infusion catheters; sensing catheters; ostomy tubes, needles, drainage tubes, biliary tubes, conduits, probes, venous access devices, medical tubing, feeding tubes, nasogastric tubes, sheath introducers; luer-lock extensions, anchor pads (securement devices), other catheters, probes and medical devices that are insertable or positionable in a subject's body and for at least one purpose selected from: infusion or administration of a substance; withdrawal or drainage of a fluid or substance, monitoring or measurement of pressure; monitoring or measurement of arterial pressure; monitoring or measurement of venous pressure; monitoring or measurement of intracranial pressure; monitoring or measurement of any physiological variable, delivery of electrical impulses or stimuli, delivery or performance of a therapy, etc.

In accordance with yet another aspect of the present invention, some or all components of the signaling system may alternatively be positioned on or in a secondary apparatus that is connected to or otherwise associated with the catheter or medical device. Examples of secondary apparatuses in which some or all components of the signaling system may be incorporated include but are not limited to: dressings, solution administration tubes, connectors, securement apparatuses, stabilization apparatuses, Stat-lock® stabilization devices (available from C.R. Bard, Inc. Murray Hill, N.J.), flush systems, an Intraflo® continuous flush system (available from Hospira, Inc., Lake Forrest, Ill.).

In accordance with yet another aspect of the present invention, there are provided systems and methods useable to facilitate removal and/or replacement of transcutaneous substance delivery systems at pre-determined times and/or upon sensing that a physiological or device-associated variable (e.g., temperature) has exceeded or is near exceeding a predetermined or calculated limit. In some such embodiments, the invention comprises a transcutaneous substance delivery system that combines a transcutaneous substance delivery device that is positionable in contact with a subject's body and is operative to deliver a substance to the subject with a signaling system that emits signal(s) which indicate at least one of; a) expiration or impending expiration of a time period and/or b) a particular change in a physiological or device-associated variable of interest.

In accordance with yet another aspect of the present invention, in embodiments where the signaling system operates to emit a signal upon expiration or impending expiration of a predetermined time period, the signal triggering apparatus may include a timer and a switch apparatus for starting the timer.

In accordance with yet another aspect of the present invention, in embodiments where the signaling system operates to emit a signal upon sensing of a predetermined change or magnitude of change in a physiological or device-associated variable, the signal triggering apparatus may comprise a sensor for sensing that variable. Examples of the types of physiological or device-associated variables that may be sensed include but are not limited to: temperature; galvanic skin response; pulse rate, disruptions in flow through a catheter or device lumen; the presence of air or bubbles in a catheter or device lumen; etc.

In accordance with yet another aspect of the present invention there is provided a method for alerting a caregiver or a subject in or on whose body a catheter or other indwelling medical device is positioned of the expiration or impending expiration of a time period and/or of a change in a physiological or device-related variable. Such methods of the present invention comprise the step of connecting or associating a signaling system of the above-summarized character with the catheter or other indwelling medical device and/or connecting or associating a signaling system of the above-summarized character to/with a secondary apparatus that is connected to or associated with the catheter or other indwelling medical device.

### BRIEF DESCRIPTION OF THE DRAWINGS

FIG. 1A is a partial perspective view of the arm of a human subject having a first embodiment of a device of the present invention, which comprises a butterfly intravenous device/timer, operatively inserted into an antecubital vein of a human subject.

FIG. 1B is an enlarged perspective view of the device of FIG. 1A.

FIG. 2A is a partial perspective view of the arm of a human subject having an intravenous cannula of the prior art inserted into an antecubital vein.

FIG. 2B is a perspective view of a second embodiment of the present invention, comprising an intravenous securement device/timer, operatively positioned to secure the intravenous cannula of FIG. 2A.

FIG. 3A is a partial perspective view of the arm of a human subject having an intravenous cannula inserted into the antecubital vein and a third embodiment of a device of the present invention, which comprises a patient bracelet/timer, positioned on the subject's wrist.

FIG. 3B is a perspective view of the bracelet device of FIG. 3A.

FIG. 4A is a partial perspective view of the arm of a human subject having an intravenous cannula inserted into the antecubital vein and a fourth embodiment of a device of the present invention, which comprises a tubing connector/timer, attached to the intravenous cannula.

FIG. 4B is a cross sectional view of the tubing connector device of FIG. 4A.

FIG. 5A is a partial perspective view of the arm of a human subject having an intravenous cannula inserted into the antecubital vein and a fifth embodiment of a device of the present invention, which comprises a tube-positionable timer, positioned on intravenous tubing that is connected to the intravenous cannula.

FIG. 5B is a top view of the tube-positionable timer of FIG. 5A.

FIG. 5C is a perspective view of a sixth embodiment of the present invention, which comprises another tube-positionable timer, positioned on a segment of intravenous tubing.

FIG. 6A is a partial perspective view of the arm of a human subject having an intravenous cannula inserted into the antecubital vein and a seventh embodiment of a device of the present invention, which comprises another tubing connector/timer, attached to the intravenous cannula.

FIG. 6B is a cross sectional view of the device of FIG. 6A.

FIG. 7A is a partial perspective view of the arm of a human subject having an intravenous cannula inserted into the antecubital vein and an eighth embodiment of a device of the present invention, which comprises a universal securement device/timer, operatively positioned to secure the intravenous cannula.

FIG. 7B is a perspective view of the device of FIG. 7A.

FIG. 7C is an electrical schematic of the device of FIG. 7A.

FIG. 7D is a circuitry diagram of the device of FIG. 7A.

FIG. 7E is a top view of a ninth embodiment of a device of the present invention, which comprises another universal securement device/timer.

FIG. 8A is a partial perspective view of the arm of a human subject having a tenth embodiment of a device of the present invention, which comprises a transcutaneous substance delivery patch/timer, positioned on the subject's arm.

FIG. 8B is a top view of the device of FIG. 8A.

### DETAILED DESCRIPTION AND EXAMPLES

The following detailed description and examples are provided for the purpose of non-exhaustively describing some, but not necessarily all, examples or embodiments of the invention, and shall not limit the scope of the invention in any way.

Certain non-limiting examples of this invention, as well as certain details regarding the acquisition, manufacture and/or structure of certain component parts of the devices of the present invention are described in the above-incorporated patent applications to which this application claims priority.

This invention provides systems and methods which measure time and/or local body temperature associated with a medical device (e.g., a catheter, transcutaneous substance delivery patch, etc.) and deliver one ore more signals indicating when some action (e.g., removal, replacement, replenishment, etc.) is impending or due based on the measured time and/or local body temperature. For example, the systems and methods of the present invention may be programmed to deliver signal(s) indicating when an intended time period (e.g., the intended time for removal, replacement or replenishment of a medical device) has expired or will expire. Alternatively or additionally, the systems and methods of the present invention may be programmed to deliver signal(s) indicating when a sensor has sensed that a sensed variable has exceeded or is close to exceeding some predetermined or calculated limit. Examples of the types of sensors and variables that may be sensed include, but are not limited to, temperature sensor(s) for sensing changes in local body temperature (as may, in some cases, be indicative of infection or localized allergic reaction), galvanic response sensor(s) for sensing changes in galvanic skin response as may be indicative of the accumulation of excessive moisture due to perspiration or entry of extraneous moisture, piezoelectric sensor(s) for sensing pulse rate as may be indicative of certain substance overdoses or untoward cardiovascular states that contraindicate further continued administration of the substance, photodetectors for sensing exposure to excessive ultraviolet or other light as may cause degradation of certain light-sensitive substances, etc.

In general, the systems of the present invention comprise electronic control circuitry 20 including components such as a microcontroller. In some embodiments the microcontroller will be continually powered (with minimal current draw until use. Other embodiments will include a switch for initiating or energizing the circuitry. The control circuitry 20 is connected to or in communication with at least a power source 22 and one or more signal emitter(s) 24a, 24b. In embodiments intended to measure time period(s), the electronic circuitry 20 will include a timer, timing standard or timing circuit. In embodiments that are intended to measure a physiological or device-associated variable, such as local body temperature, the system will include a sensor 18 for sensing the desired physiological or device-associated variable (e.g., local body temperature) and transmitting a signal indicative of the sensed variable to the control circuitry 20. Embodiments of the invention may measure time periods only, physiological or device-associated variable(s) only or both time period(s) and physiological or device-associated variable(s).

The signal emitter(s) may comprise light emitters, such as LEDs, liquid crystal display(s), sound emitter(s), mechanical or piezoelectric beeper(s), vibrator(s), etc. In some embodiments, the signal emitter(s) may comprise one or more LEDs and the desired signals may be displayed using different patters or colors of LED flashes, thereby simplifying the device and minimizing power consumption. For example, in systems which measure a time period, LEDs may emit a first pattern and/or color of LED flashes to indicate when the system has been initiated or energized to begin timing of the time period, a second pattern or color of LED flashes to warn when the time period is nearing expiration and a third pattern or color of LED flashes to indicate when the time period has expired. If such system were to further include an optional sensor 18 for sensing a physiological or device-associated variable(s), yet another color or pattern of LED flashes may indicate when the sensed variable has exceeded a predetermined limit programmed into the control circuitry 20.

In some embodiments of the invention, at least some components of the system, such as the electronic circuitry 20, power source 22 and any optional sensor 18, may be encapsulated or integrated within a catheter, transdermal substance delivery patch or other medical device in a manner described in the above-incorporated U.S. patent application Ser. No. 12/322,010, the entire disclosure of which is hereby incorporated herein by reference.

The power source 22 may be any battery of suitable size and power output, such as either a film battery or button-type lithium battery. Examples of commercially available batteries useable in at least some of the embodiments described herein are 3.0 volt lithium ion non-rechargeable batteries sold under product designations CR1220, CR1025, and CR925 by a number of purveyors, including Harding Energy, Inc. The components of the system may be mounted on or incorporated into the material (e.g., plastic material) of a medical device such as a catheter or transdermal substance delivery patch. The term “catheter” as used herein shall be broadly construed to include but not necessarily be limited to intravenous (IV) or intra-arterial catheters, insertion needles, sheaths, needle/sheath combinations, other cannulae, IV infusion sets; arterial lines; connectors, twist connectors, snap-in connectors, insertion needle holders; securement devices (such as Statlock® stabilization devices available from C.R. Bard, Inc.), plastic tabs or “butterfly” wings of an IV cannula, Foley catheters and other bladder catheters, intracranial catheters, pressure monitoring catheters, etc. The term “transdermal substance delivery patch” as used herein shall be broadly construed to include, but not necessarily be limited to, any apparatus that is positionable on the body of a human or animal body to transcutaneously deliver a drug or other substance, such as single-layer drug-in-adhesive patches, multi-layer drug-in-adhesive patches, reservoir patches, matrix patches and vapor patches. The term “substance” as used herein shall be broadly construed to include, but not necessarily be limited to, any feasible drugs, prodrugs, proteins, gene therapy preparations, cells, diagnostic agents, contrast or imaging agents, biologic agents, therapeutic fluids or solutions, etc. Such substances may be in bound or free form, liquid or solid, colloid or other suspension, solution or may be in the form of a gas or other fluid or non-fluid.

Time Measurements: In some embodiments which measure time period(s), the electronic circuitry 20 will be switched on or energized when the time period is to begin, such as when a catheter or other indwelling tube or device is inserted into the subject or when a transdermal substance delivery patch is applied to the subject's skin. This will cause a timing device and microcontroller included in the circuitry 20 to begin a count-down or count-up sequence. During the time between activation and completion of the timing interval, an LED may periodically flash to notify personnel that the device is working. At the end of the predetermined time interval, a single or a series of LED flashes, sounds or vibrations, may indicate to the patient or health provider that the time period has expired and, thus, it is time to remove or change the device. In some embodiments, if the device remains in or on the subject's body past the end of the predetermined time period, the electronic circuitry 20 may be further programmed to cause the signal emitter to emit further or different signals (e.g., flashes, sounds or vibrations) to indicate how much time has elapsed since the end of the time period.

Temperature Measurement: In some embodiments which incorporate a sensor 18, that sensor may measure local body temperature, such as skin surface temperature, at the insertion or placement site of the device. In such embodiments, the microcontroller of the electronic control circuitry 20 may be programmed to compute and store average sensed temperatures over certain periods of time. For example, the device might (depending on user request) measure skin temperature for periods of 20 minutes. At the end of each 20 minute period, the skin temperature multiplied by the time period (time-temperature product) is stored into the memory of the microcontroller of the control circuitry 20. If at any time in the future the average skin temperature (time-temperature product) during any 20 minute interval rises above the “baseline,” the signal emitter will emit a signal such as light flash(s), sounds or vibrations. Optionally, the microcontroller may also be programmed to cause the signal emitter to emit a signal indicating the magnitude of temperature change sensed by the sensor 18. For example, a 1 degree Celsius rise in temperature may result in a red LED flashing once every 5 seconds. A 2 degree Celsius rise may result in 2 flashes of the red LED every 5 seconds. By “integrating” the temperature over time (that is, the area-under-the-curve of temperature versus time—referred to as time-temperature product) false triggering of a temperature warning signal may be averted. Furthermore, as there is no attempt to correlate skin temperature with core body temperature, inaccuracies associated with peripheral vasoconstriction, dilation, or clothing insulation will be minimized or eliminated. Unlike previous approaches that utilize two or more temperature sensors, this invention can, in at least some embodiments, accomplish the task through the use of a single temperature sensor 18 through the novel approach of “integrating over time” the area-under-the-curve of temperature versus time. In such single-sensor 18 embodiments, the sum of the product of temperature measurements over a period of time will determine whether an alert will be activated. This approach allows for 1) the use of a single sensor at lower cost and 2) eliminates false trigger owing to slight variations in skin temperature. Information may be conveyed to the patient or clinical personnel using any suitable signal emitters, such as flashes of light, audible beeps or vibrations.

### EXAMPLE 1

### Intravenous Cannula System

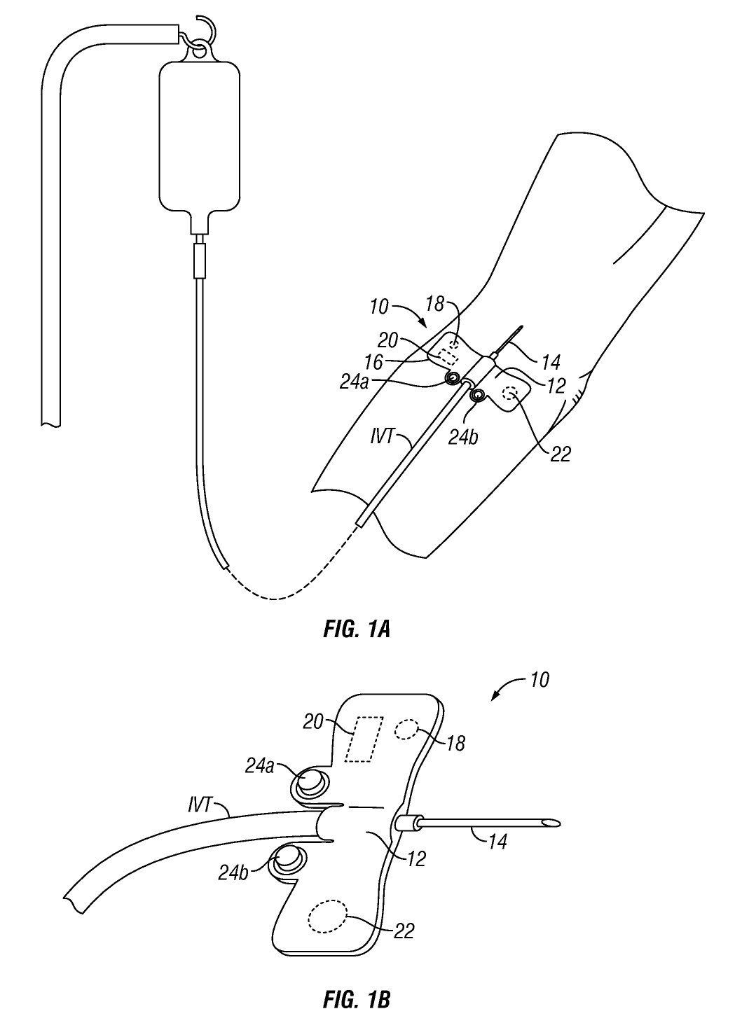

FIGS. 1A-1B show an example of a butterfly intravenous cannula system 10 of the present invention. This butterfly intravenous cannula system 10 comprises an intravenous cannula 14 (e.g., a needle, sheath or needle/sheath combination) with a winged hub assembly 12 on its proximal end. This butterfly intravenous cannula system 10 is connectable to an intravenous tube IVT for administration of intravenous solution(s) through the cannula 14. Embedded within or mounted on the winged hub assembly 12 are the electronic control circuitry 20, power source 22, an optional sensor 18 for sensing a physiologic or device-related variable (in this case the temperature of the subject's skin) and first and second signal emitters 24a, 24b. In this example, the first signal emitter 24a is a green LED and the second signal emitter 24b is a red LED. The control circuitry 20 includes at least a microcontroller having a timing circuit and, optionally, also programmed to monitor and process a measured body or device-associated variable, such as body temperature, based on signals received from the sensor 18. The control circuitry 20 may be connected to the power source 22 at the time of manufacture or it may include a switch that is triggerable by the user to energize the circuitry 20, such as by removal of a pull-out tab that allows electrical contacts to abut one another, compression of a press-in switch twisting of a twistable switch (as described below) or other suitable switching apparatus, to power up the control circuitry. Devices in which power from the power source 22 flows to the control circuitry continually from the time of manufacture may have somewhat lesser potential shelf life due to minimal power draw by the microcontroller or other components of the control circuitry 20. Thus, in embodiments where it is desired to maximize shelf life of the device, it will typically be desirable to include some type of switch to allow the user to volitionally power up the circuitry 20 at the time of use. After the control circuitry 20 has been energized and/or after timing of a time period has initialized, the control circuitry 20 may, in this example, cause the first signal emitter 24a to emit a signal indicating successful energization and/or actuation, such as one or more series of rapid green LED flashes. Thereafter, during the timing of a first time period, the control circuitry 20 will cause the first signal emitter 24a to emit occasional, brief green LED flashes to indicate that the device is operating within acceptable parameters. Upon expiration of a first time period, the control circuitry 20 will cause the first and second emitters 24a, 24b to alternately emit red and green LED flashes indicating that a time for intended removal or replacement of the IV cannula 14 is approaching. Optionally, the frequency of the alternating red/green LED flashes may increase as the deadline draws nearer. Upon expiration of a second time period, the control circuitry 20 will cause the first signal emitter 24a to remain off and the second signal emitter 24b to emit red LED flashes indicating that the time for intended removal or replacement of the IV cannula 14 has expired. Optionally, the frequency of the red LED flashes may increase as the time for intended removal or replacement of the IV cannula becomes further past due. The number of LEDs may vary from 1 to several. For example, in some embodiments, three LEDs (e.g., red, green and yellow) may be provided and one of the LEDs (e.g., the yellow one) may be caused to flash separately to indicate a specific warning (such as when the intended time period is close to expiration but has not yet actually expired).

The sensor 18 in the particular example of FIGS. 1A-1B comprises a thermistor that senses changes in the temperature of the patient's skin surface underlying the tab or wing of the winged hub assembly 12 in which the sensor 18 is mounted. The microcontroller of the control circuitry 20 receives signals from the sensor and may compute and store average temperature per unit of time as described above. If the sensed temperature (or average temperature per time period) exceeds a preset limit, the control circuitry 20 will cause the first signal emitter 24a to remain off and the second signal emitter 24b to emit red LED flashes in a unique pattern indicating that a local body surface temperature limit (e.g., 38 degrees C.) has been exceeded. This may signal to the subject and care givers that an infection or local inflammation has begun to occur at the site of the intravenous cannula 14 insertion, thus enabling the cannula 14 to be removed and replaced before a more serious infection or sepsis occurs.

Of course, this example is non-limiting. Those of skill in the art will appreciate that a single LED may be used instead of multiple LEDs, LED(s) of a single color may be used instead of red and green LEDs and various alternative flashing or signaling schemes may be used.

### EXAMPLE 2

### Intravenous Securement Device

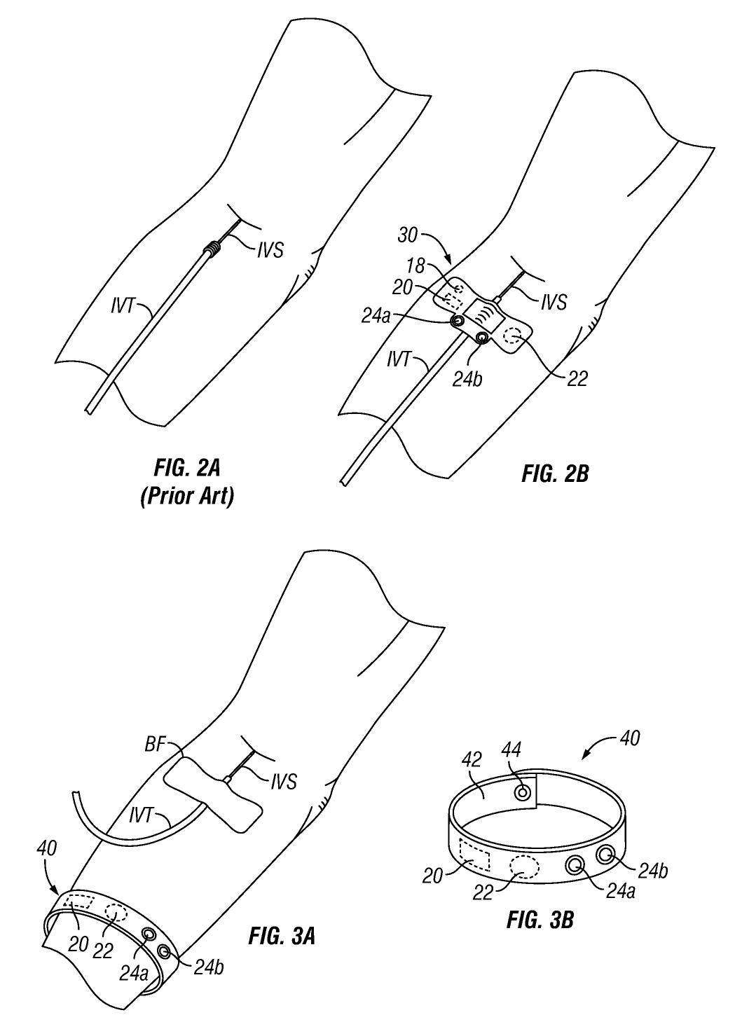

FIG. 2A shows an intravenous sheath IVS inserted into a subject's body and connected to an intravenous tube IVT for delivery of intravenous fluids or solutions through the intravenous sheath IVS. FIG. 2B shows a securement system 30 of the present invention applied to the subject's body to secure and hold the intravenous sheath IVS in place. This securement system 30 comprises a structure, such as a molded plastic fixture, that engages or grasps the exteriorized hub of the intravenous sheath IVS and outwardly extending wings or tabs that have adhesive undersurfaces which adhere to the subject's skin. Incorporated within and/or mounted on the structure and/or wings/tabs are the same control circuitry 20, power source 22, optional sensor 18 for sensing the temperature of the subject's skin and first and second signal emitters 24a, 24b as described above in Example 1.

The configuration, shape and structure of the securement system 30 of the present invention may vary such that it may be used for securing of various types of cannulae, sheaths and catheters other than intravenous sheaths IVS of the type shown in this non-limiting example. Examples of alternative configurations for this securement system 30 include those of the Statlock® Stabilization Devices including but not limited to the Statlock® Universal Plus Stabilization Device; Statlock® Stabilization Devices for PICC and Central Line Catheters; Statlock® PICC Stabilization Device; Statlock® PICCII Stabilization Device; FIRST Statlock® PICC Stabilization Device; Statlock® CV Plus Stabilization Device; Statlock® CV Ultra Stabilization Device; Statlock® CVHuber Plus Stabilization Device; Statlock® Dialysis Stabilization Device; Other Statlock® Dialysis Stabilization Devices; Statlock® Dialysis Stabilization Device for Fixed Wing Catheters; Statlock® Dialysis Stabilization Device for Attachable Wing Catheters; Statlock® Dialysis II Stabilization Device; Statlock® IV Ultra Stabilization Device and Statlock® Foley Stabilization Device, which are commercially available from C.R. Bard, Inc., Murray Hill, N.J.

### EXAMPLE 3

### Patient Bracelet

FIGS. 3A and 3B show a patient bracelet 40 of the present invention. This patient bracelet comprises a wristband 42 having a connector 44 for attaching the wristband 42 around a patient's wrist. The wristband 42 may be imprinted with patient identification information in the manner typical of hospital ID bracelets. Additionally, incorporated within and/or mounted on the structure and/or wings/tabs are the same control circuitry 20, power source 22 and first and second signal emitters 24a, 24b as described above in Example 1. In this example, the bracelet 40 is shown on the arm of a subject in whom a standard intravenous sheath IVS with butterfly wings BF is inserted and to which an intravenous tube IVT is connected. The bracelet 40 may be programmed to signal the expiration or impending expiration of a time period for removal or replacement of the intravenous sheath IVS in the manner described above. However, it is to be appreciated that this bracelet 30 has universal timing utility and may be used for timing various time periods other than that for removal of replacement of an intravenous sheath IVS. Also, in pediatric settings, fear and apprehension experienced by young patients may be mitigated by offering them a flashing LED hospital bracelet.

### EXAMPLE 4

### Intravenous Tubing Connector Bridge

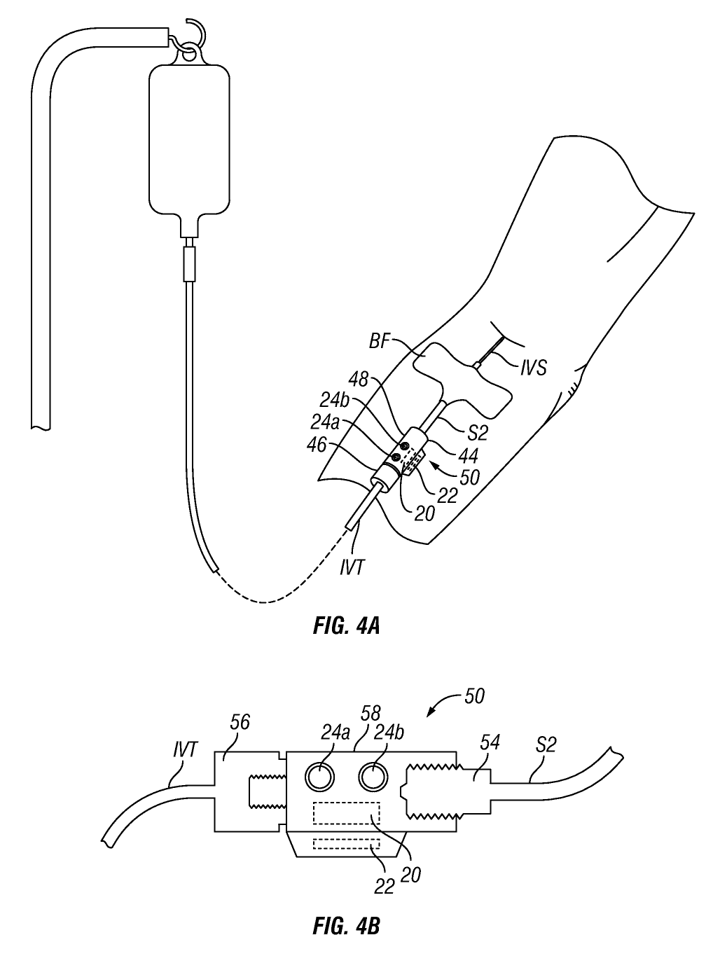

FIGS. 4A and 4B shows an example of an intravenous tubing connector system 50 of the present invention. This system 50, comprises a flow-through member 58 that is connectable between standard male 54 and female 56 tubing connectors. In this non-limiting example, the male connector 54 is on the proximal end of an intravenous cannula device S2 that comprises an intravenous sheath IVS and the female connector 56 is on the distal end of an intravenous solution administration tube IVT, or vice versa. The male connector 54 connects to one end of the flow-through member 58 and the female connector 56 connects to the other end of the flow-through member 58. Intravenous fluid or solutions flow from the intravenous tube IVT, through a bore within the flow-through member 58 and then into the intravenous cannula device S2. Incorporated within and/or mounted on the flow-through member 58 is the same control circuitry 20, power source 22 and second signal emitters 24a, 24b as described above in Example 1. The control circuitry 20 is programmed to signal expiration or impending expiration of a time period for intended removal or replacement of the intravenous cannula system S2. This embodiment of the invention may be used with standard, commercially intravenous tubing sets and cannulae without modification of those standard devices. This approach provides for a universal connection and insertion device, capable of being used on all IV, intra-arterial, intracranial, intra cavity tubing. The invention may also be encapsulated directly into the connector of existing IV tubing sets, central artery or venous sets, etc.

### EXAMPLE 5

### Tube-Mountable Ring or Clip

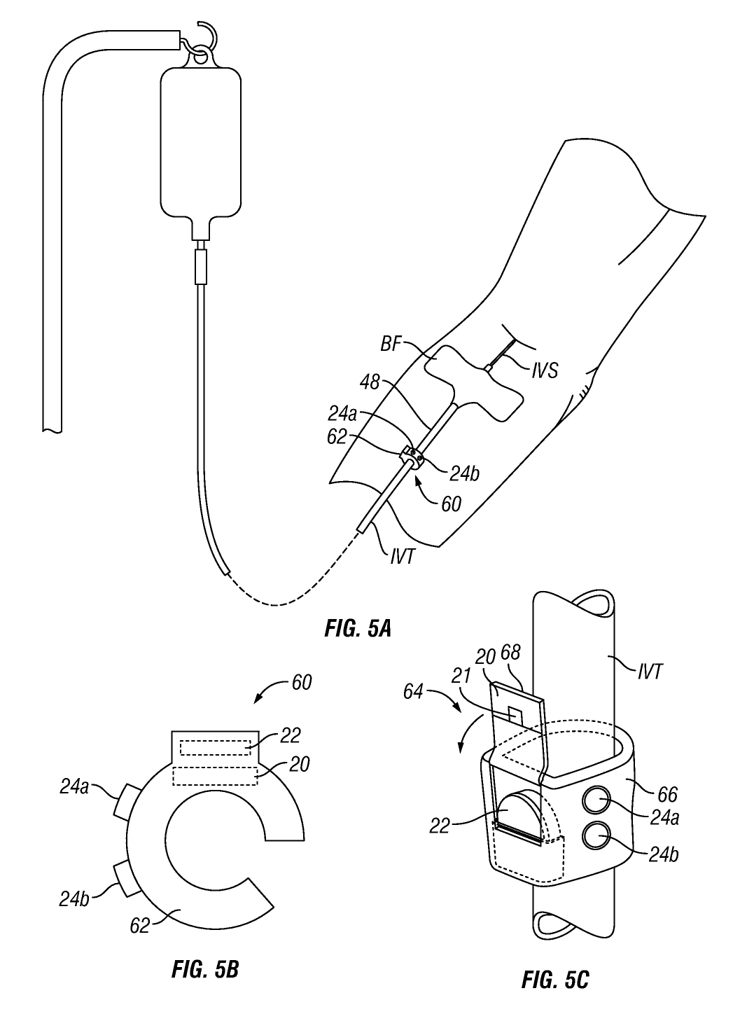

FIGS. 5A through 5C show embodiments of the invention wherein a timing and signaling system is incorporated into a “ring” or “clip” that is attached to, mounted on or integrally formed on a tube or tubing connector. In cases where these embodiments are attachable to a standard tube or tubing connector, they may not require any modification of the standard tubing or tubing connector, thereby minimizing the need for regulatory evaluation and allowing for use with IV sets already approved by the appropriate regulatory agency.

Specifically, FIGS. 5A and 5B show a clip/timer system 60 that comprises a clip 62 that is attachable to a tube 48 which, in this non-limiting example, leads to an intravenous sheath IVS that has been inserted into a subject's body. Incorporated within and/or mounted on the clip 62 are the same control circuitry 20, power source 22 and signal emitters 24a, 24b as described above in Example 1. In this example, the control circuitry 20 is programmed to signal expiration or impending expiration of a time period for intended removal or replacement of the intravenous sheath IVS, as shown. However, it should be appreciated that this clip/timer system 60 may be attached to any tubing or various other items and may be used for timing of a variety of time periods relating to various types of medical devices.

FIG. 5C shows a ring/timer system 64 that comprises a ring 66 having a door 68. The ring 66 is passable around a segment of tubing, such as the intravenous tubing IVT shown in this example or may be formed as part of tubing or a connector at the time of manufacture. In some embodiments, the ring 66 may have a tube grasping structure, such as am elastomeric liner, which will frictionally engage a tube that the ring 66 is mounted on, thereby holding the ring 66 in a substantially stable position on the tube. In some embodiments, the ring 66 may be flexible and may have a slit in it to allow it to be passed around a tube. When it is desired to energize the control circuitry 20 and commence timing of the time period, the door 68 is closed causing contact 21 to contact and complete a circuit with the power source 22 (e.g., battery). Incorporated within and/or mounted on the ring 66 and/or door 68 of this example are the same control circuitry 20, power source 22 and signal emitters 24a, 24b as described above in Example 1. In this example, the control circuitry 20 may be programmed to signal expiration or impending expiration of a time period for intended removal or replacement of an intravenous cannula of any type to which the intravenous tube IVT is connected. However, it is to be appreciated that this ring/timer system 60 may be attached to any tubing and various other items and may be used for timing of a variety of time periods relating to various types of medical devices.

### EXAMPLE 6

### Tubing Connector

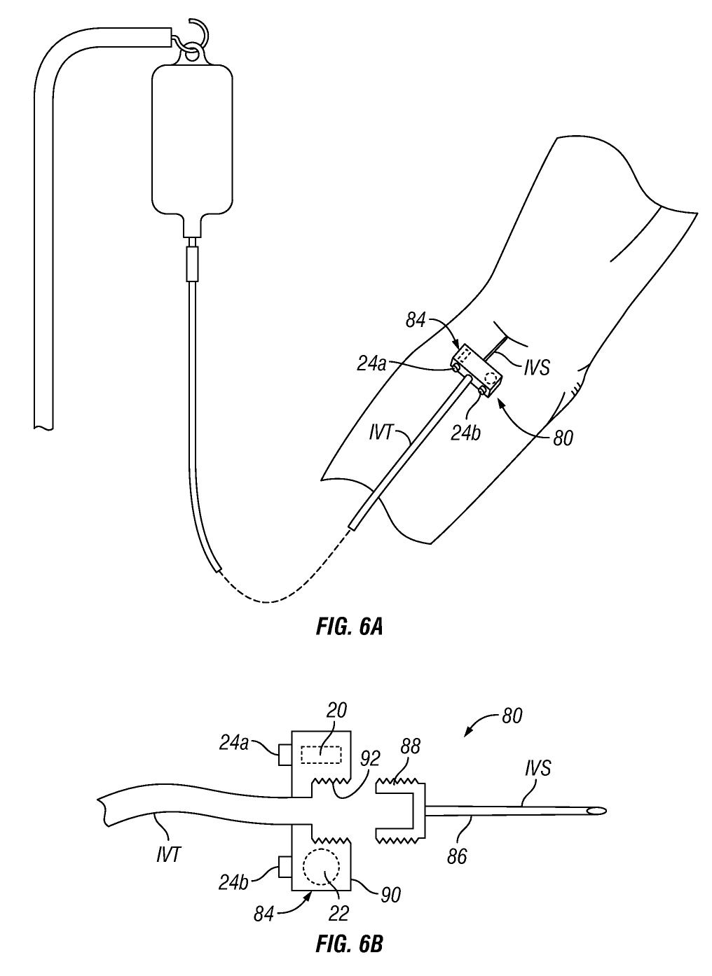

FIGS. 6A and 6B show a tubing connector system 80 of the present invention which comprises a connector member 84 which has, incorporated therein and/or mounted thereon, control circuitry 20, power source 22 and signal emitters 24a, 24b as described above in relation to Example 1. In the particular example shown, the connector member 84 is a female connector member located on the end of an intravenous tube IVT and connectable to a male connector 88 on the proximal end of an intravenous cannula device comprising a standard intravenous sheath IVS. However, the control circuitry 20, power source 22 and signal emitters 24a, 24b may alternatively be incorporated into the male connector 88, or any other type of tubing connector.

### EXAMPLE 7

### Universal Clinical Timers with Multiple Time Period Options

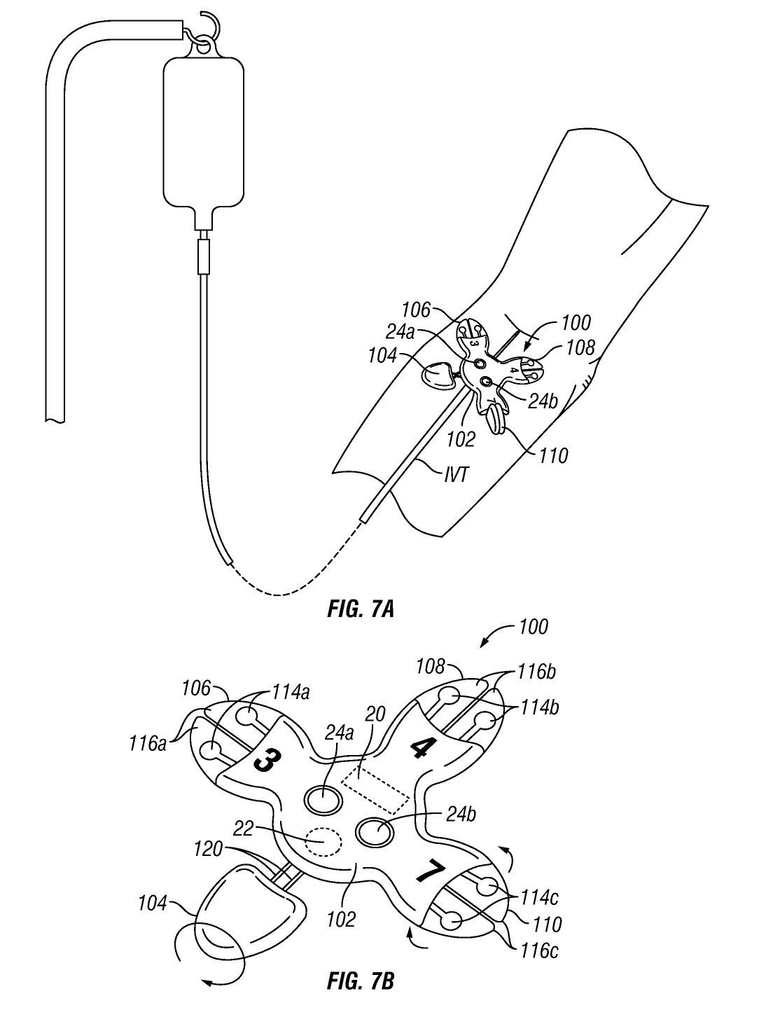

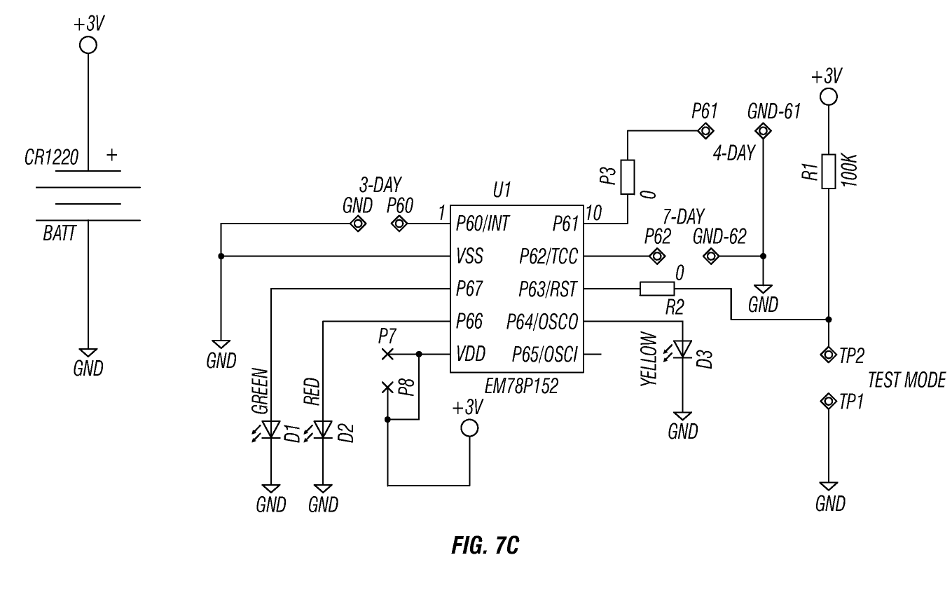

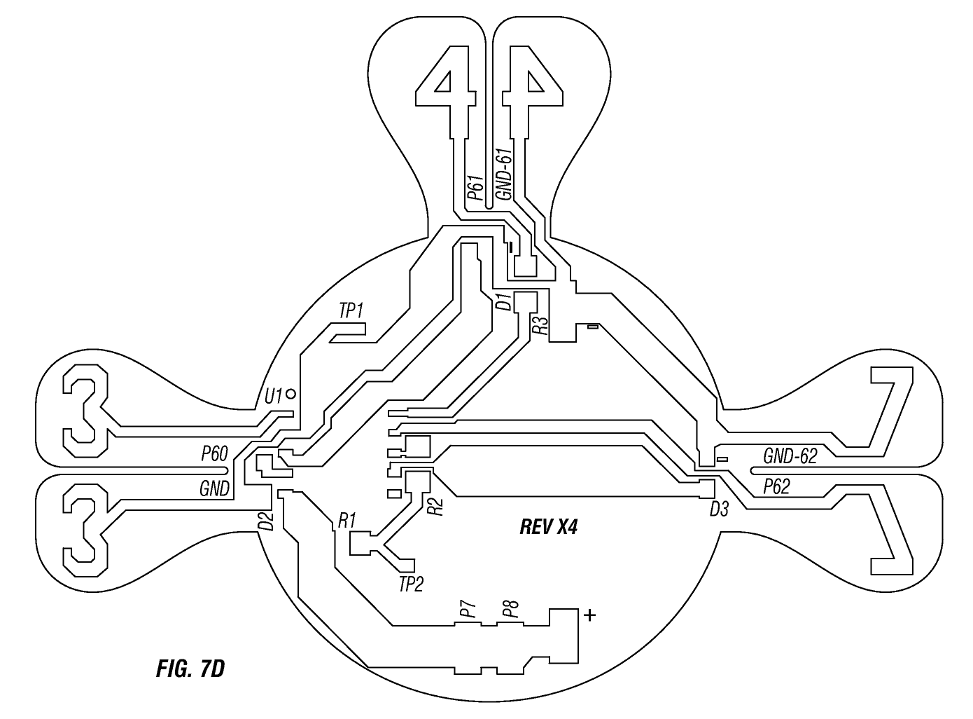

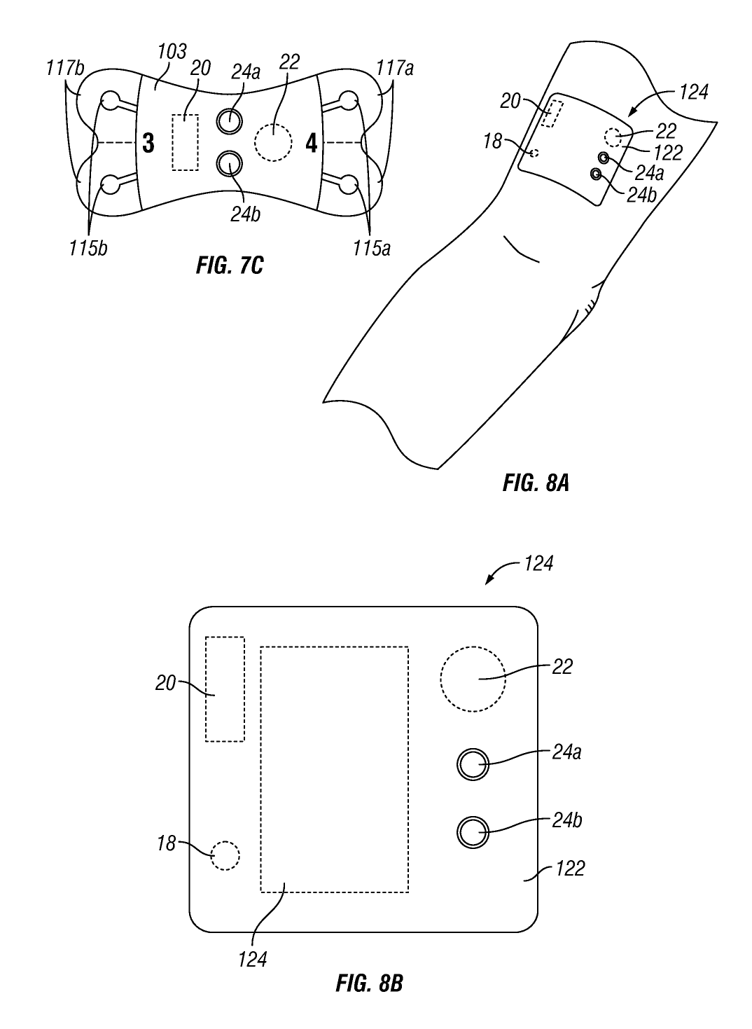

FIGS. 7A-7D show a clinical timing system 100 that may optionally have an adhesive surface allowing it to be adhered to a subject's skin. This system 100 is useable in many applications including, but not limited to, use as an IV securement device in the manner depicted in FIG. 7A.

This system comprises a multi-lobed body 102 having electrical control circuitry 20 comprising a microcontroller, a power source 22 and signal emitters comprising green and red LEDs 24a, 24b, as described above. Circuit diagrams for the electrical control circuitry 20 and related contacts is shown in FIGS. 7C and 7D. The components of the circuit, as shown in the diagrams are as follows:

| | | Part Number/ | |
|---|---|---|---|
| Component | Description | Package | Manufacturer |
| U1 | | EM789152S | Elan |
| (Microcontroller) | | | Microelectronics |
| D1 | Green LED | 19-213SYGC/ | Everylight or |
| | | S530-A4/TR8-0603 | equivalent |
| D2 | Red LED | 19-213SURC/ | Everylight or |
| | | S530/A3/TR8-0603 | equivalent |
| D3 | Yellow LED | 19-213UYC/ | |
| (Optional item | | S530-A2/TR8-0603 | |
| 24c—not shown | | | |
| on FIGS. 7A-7B) | | | |
| Battery | 3.0 V | CR1220 | Harding Energy |
| | Lithium Ion | | |
| Battery Tabs | | | Harding Energy |
| Circuit Board | | Ink Jet Flex | Ink Jet Flex |

Numerous variations of and/or additions to this circuit are possible within the scope of the present invention. For example, optionally, in some embodiments, the circuit may include a capacitor (e.g., 0.01uf-10 #603). A twistable on-off tab 104 is connected to wires 120. Lobes 106, 108 and 110 have pinchable contact tabs 116a, 116b, 116c with electrical contacts 114a, 114b, 114c on their upper surfaces. Each lobe is labeled with a particular time period, i.e., lobe 106 is for a 3 day time period, lobe 108 is for a 4 day time period and lobe 110 is for a 7 day time period.

Initially, when it is desired to energize the system, the user twists tab 104 causing wires 120 to contact one another, thereby completing a circuit which connects the battery to the electronic control circuitry 20 which may be programmed to cause the first (green) LED 24a to emit flash(es) to confirm that the system has been energized. Thereafter, the user selects a particular time period to be timed and pinches the appropriate contact tabs together to cause that tab's contact surfaces to abut. For example, in FIG. 7A the 7 day time period has been selected and the tabs 116c of the third lobe 110 have been pinched together, causing contacts 114c to abut. This completes a circuit which triggers the electrical control circuitry 20 to commence timing of the 7 day time period. The electrical control circuit 20 will then cause the LEDs 24a, 24b to flash signals indicating that the time period is progressing, near expiration or expired, as described in Example 1 above. Optionally, in embodiments such as this where the user selected from a plurality of available time periods, the confirmatory signals flashed by the first (green) LED 24a at the commencement of and during timing of the time period may be specific to that selected time period, i.e., one flash pattern will signal if the 3 day period is being timed, another flash pattern will signal if the 4 day time period is being timed and yet another flash pattern will signal if the 7 day time period is being timed.

These devices may be designed to offer any feasible number of available time periods. For example, FIG. 7E shows a simpler variation of a universal clinical timer device 103 wherein only two time periods, 3 days or 4 days, are provided for. In this embodiment, the electronic circuitry 20 may always be energized or it may include any suitable switch (e.g., a pull-out tab that allows contacts to come together, a compressible switch or a twist tab as shown in the device 100 of FIG. 7A). The user will then pinch together either the 4 day tab members 117a causing contacts 115a to meet and commence timing of the 4 day period or the 3 day tab members 117b causing contacts 117b to meet and commence timing of the 3 day period. The signaling members 24a, 24b, such as LEDs, will then emit signal(s) in any of the manners described above with respect to the other examples. Of course, the 3 and 4 day periods are merely examples. Any suitable time periods may be used.

As this device may be incorporated into a disposable component of a dressing or infusion set, cost of production is of paramount importance. Furthermore, size dictates a small power source. In one embodiment the power source is a small, button-sized lithium battery, although other energy sources would be usable. The microcontroller can be programmed to be in “sleep mode” most of the time, but despite extremely low power consumption, shelf-life of the device will be compromised if the battery is continuously activating the circuitry. Accordingly, an on-off switch should be incorporated. This switch must be low cost, of small size, and easy to use. Commercially available switches do not meet these requirements. Tactile and dome switches are momentary in nature. They cannot create a permanent connection. One approach is to insulate the battery from the circuitry using a piece of non-conductive paper. Upon removing the paper the battery comes into contact with the circuit thereby activating the device. Inserting paper between the battery and contact is labor intensive in large volume production. A novel approach that lends itself to volume production and is low-cost and easy to use is illustrated in FIGS. 7A and 7B (although the particular shape of the dressing is optional). The battery 22 may be insulated from the remainder of the circuit by the two fine wires 120 that can be brought in contact to complete the circuit by twisting the twist tab 104.

In some embodiments, the present invention may operate to alert clinical personnel that a period of time has elapsed and a change in insertion site of an indwelling catheter or IV or arterial line is indicated, or that the temperature of the skin at such insertion point is elevated warning that an infection may be present. The inventions represent an improvement on existing intravenous or intra-arterial insertion needles, catheters, connectors, or securement devices as well as an improvement on existing bladder or intracranial insertion catheters. By providing a level of “intelligence” into the infusion or drainage set of these devices, additional safeguards are introduced, thereby providing opportunity for improved quality of care and outcomes.

### EXAMPLE 8

### Transcutaneous Substance Delivery Patch

The present invention also provides transcutaneous substance delivery patches that have, incorporated therein or thereon, timing and/or sensing systems in accordance with this invention. Delivery of medication across the skin is dependent on many variables, but two key concerns are the duration of contact with the skin, and the temperature of the skin-patch interface. Increasing temperature at the skin-patch interface increases blood flow with consequent alteration of the drug-delivery pharmacokinetics. Pharmaceutical manufacturers, currently incapable of adjusting for temperature, must strike a balance between the therapeutic index (safe versus toxic levels of a drug) and the efficacy of the agent at the dose delivered. More accurate dosing can be accomplished if the time of contact with the skin is adjusted according to the average temperature of the skin contact area. For example, if the subject experiences a fever or local irritation owing to the patch adhesive, absorption of the medication would be enhanced. If the time duration of the application of the patch were shortened, then the total dose delivered could be held constant. Stated in an alternative way, the product of time and temperature (area-under-the-curve) could be held constant. As temperature correlates with transdermal delivery, the total dose delivered would be regulated independent of the temperature variable.

In many situations the therapeutic index may be sufficiently high that temperature compensation is not necessary. Nonetheless, removal of the patch at an accurate hourly time (versus day) would enhance the confidence that the total dose delivered was appropriate. A device that provides a visible or audible alert relating to the length of time that the patch was applied would allow for much more accurate dosing of a drug.

The transdermal substance delivery patches of this invention provide electronic and microcontroller control of measurement of time and/or skin temperature and/or may calculate the appropriate duration of application of a medication patch based on sensed conditions such as temperature. The status of such measurement can be visually displayed using Light Emitting Diodes (LEDs) or Liquid Crystal Display, and can be audibly provided using a mechanical or piezoelectric beeper. The wearer may be notified via a vibration device on the patch. Furthermore, information may be displayed using a pattern of LED flashes, thereby simplifying the device and minimizing power consumption. The device is battery powered, using either a film battery, button-type lithium battery, or other power source. The invention is incorporated into the patch and the display is visible to the subject. By incorporating the device directly into the body of the patch, the subject, clinical personnel or care givers need not maintain time or temperature measurement.

Some transcutaneous substance delivery systems of the present invention will signal a user when a predetermined or calculated time period has expired (e.g., when it is time to remove or replace the transcutaneous substance delivery system). In these embodiments, the transcutaneous substance delivery system includes a timing device and a signaling device (e.g., visual and/or audible signal emitting apparatus). The timing device times a desired time period during which the transcutaneous substance delivery system is intended to remain in place on the subject's body. Upon expiration of that desired time period, and optionally at one or more warning time points preceding its expiration, the timer causes the signaling apparatus to provide a signal to the user. One non-limiting example of this embodiment of the invention is a timer-equipped transdermal substance delivery patch which is applied to the skin of the subject. In this example, the timing device and a microcontroller begin a count-down or count-up sequence when the transdermal substance delivery patch is applied to the subject's body. At the end of the predetermined or calculated time interval, a single or a series of LEDs will begin to flash indicating to a user (e.g., the subject or health provider) that the time has arrived to remove or change the patch. During the time between activation and completion of the timing interval, an LED may periodically flash to notify personnel that the device is working. At the end of the period a sequence of flashes will convey timing information, such as how many hours have elapsed since the end of the timing period. Alternatively, the wearer may be notified of the time to remove the patch via an audible signal or a vibration device.

In addition or alternatively to the timing apparatus described in the example above, the transcutaneous substance delivery systems of the present invention may incorporate sensing apparatus which will sense a particular physiological or device-associated variable and will cause the signaling system to issue signal(s) to alert a user when the sensed variable has exceeded or is close to exceeding some predetermined or calculated limit. Examples of the types of sensors and variables that may be sensed include, but are not limited to, temperature sensor(s) for sensing changes in temperature, galvanic response sensor(s) for sensing changes in galvanic skin response as may be indicative of the accumulation of excessive moisture due to perspiration or entry of extraneous moisture, piezoelectric sensor(s) for sensing pulse rate as may be indicative of certain substance overdoses or untoward cardiovascular states that contraindicate further continued administration of the substance, photodetectors for sensing exposure to excessive ultraviolet or other light that may cause degradation of certain light-sensitive substances, etc.

FIGS. 8A and 8B show one non-limiting example of a substance delivery patch 124 of this invention. This substance delivery patch 124 comprises a patch body 122 having control circuitry 20, power source 22 and signal emitters 24a, 24b, a body temperature sensor 18 and a substance eluting reservoir 124 incorporated therein or positioned thereon. When this substance delivery patch 124 is applied to the skin of the subject, a triggering switch (not shown) is used to turn on or energize the electrical control circuitry 20. Thereafter, the substance passes from the reservoir 124 through the skin at the rate determined by the particular construction of the patch and, at the same time, the temperature sensor 18 begins measuring the skin temperature below the patch body 122 or a temperature of the patch itself. This measurement may represent an average temperature during a period of time. For example, the device might (depending on manufacturer's request) measure “baseline” skin temperature for 20 minutes. Should an infection or inflammation of the skin below the patch or any other factors such as sun exposure, exercise, etc., result in a rise in temperature of the skin, the device will calculate the total number of minutes that the temperature is elevated, and the difference between baseline and the new temperature (hereinafter referred to as the “delta temperature”). This delta temperature will be used to arrive at a time-temperature product and will be stored in memory. When the time-temperature product reaches the predefined threshold stored in memory, the alert will be activated. For example, if the skin under the patch never experiences a rise in temperature, then the time-temperature product will be zero, and the interval will be that set at the factory. If, however, the temperature rises by 1 degree, then the 1-degree delta multiplied by the running-time that the patch has been applied will yield a product that shortens the recommended period that the patch should be worn. The alert device will notify the subject to remove the patch at a time somewhat earlier than anticipated. The relationship between delta rise of temperature and time-shortening may be linear or non-linear, and the compensation equation will be stored in the microcontroller. The visual LED will also provide indication of the magnitude of rise in temperature. For example, a 1 degree Celsius rise in temperature can result in a red LED flashing once every 5 seconds. A 2 degree Celsius rise can result in 2 flashes of the red LED every 5 seconds. By “integrating” the temperature over time (that is, the area-under-the-curve of temperature versus time—referred to as Time-Temperature Product), the device eliminates false triggering. Furthermore, as there is no attempt to correlate skin temperature with core body temperature, inaccuracies associated with peripheral vasoconstriction, dilation, or clothing insulation are eliminated. Unlike previous approaches that utilize two or more temperature sensors, this invention accomplishes the task through the use of a single temperature sensor. It does so through the novel approach of “integrating over time” the area-under-the-curve of temperature versus time. Hence, it is the sum of the product of delta temperature measurements over a period of time that determines whether an alert will be activated, and how the total time that the patch is worn will be adjusted. This approach allows for 1) the use of a single sensor at lower cost and 2) eliminates false trigger owing to slight variations in skin temperature.

It is well recognized that absorption of medication through the skin is time and temperature dependent. In this example, the device provides a means to compensate for changes in temperature of the skin beneath the patch, and to adjust the total time that the patch is worn. Should the temperature of the skin below the patch rise, the device calculates the new time-temperature product and alerts the wearer to remove the patch at an elapsed time earlier than anticipated. The same will occur should the skin temperature decrease, albeit the duration of application of the patch to the skin will be lengthened. While the above discussion uses temperature as an example, other physiologic variables will alter the rate of absorption. These variables include, but are not limited to pH, galvanic skin response, conductivity of the skin, moisture detection, pulse rate (detected using piezoelectric devices), etc.

In this particular example, the entire circuitry and battery can be encapsulated within the transdermal medication patch body 122 in ways known in the art or as described in Applicant's above-incorporated U.S. patent application Ser. No. 12/322,010.

FIGS. 8A and 8B show one of many possible examples of a transcutaneous substance delivery system of the present invention. In this example, the transcutaneous substance delivery system comprises a reservoir-type transdermal patch. However, as those of skill in the art will appreciate, the present invention may alternatively be incorporated into any of the other types of transcutaneous substance delivery systems.

This substance delivery patch 124 may operate to compensate delivery time and the duration of time that a medication patch must be worn based on temperature and/or other physiological variable(s) of the skin below the patch 124 or other physiological variable such as heart rate or other systemic cardiodynamics, and to notify the wearer that it is time to remove the medication patch. The temperature of the skin below the patch or other physiological variable is monitored, and the total time that the patch is worn is diminished or lengthened based on the well recognized relationship between rate of medication absorption through the skin and skin temperature or physiologic variable. The wearer is notified of the status of the temperature, physiologic variable, and time via visual, vibration and auditory means.

It is to be appreciated that the invention has been described here above with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with any other embodiment or example, unless otherwise specified or unless doing so would render the other embodiment or example unsuitable for its intended use. Also, where the steps of a method or process have been described or listed in a particular order, the order of such steps may be changed unless otherwise specified or unless doing so would render the method or process unworkable for its intended purpose. All reasonable additions, deletions, modifications and alterations are to be considered equivalents of the described examples and embodiments and are to be included within the scope of the following claims.

What is claimed is:

1. A signaling system associated with a catheter, infusion or drainage tubing, transdermal substance delivery patch or other medical device positioned on or in the body, comprising:
a signaling apparatus; and
a signal triggering apparatus which causes the signaling apparatus to emit a signal upon occurrence of at least one event selected from: a) expiration or impending expiration of a predetermined time period and b) a change in a physiological or device-associated variable.

2. A system according to claim 1 wherein the signaling apparatus is selected from the group consisting of: visual signaling apparatus, light emitting apparatus, light emitting diodes, auditory signaling apparatus, sound emitting apparatus, tactile signaling apparatus, vibrators and shakers.

3. A system according to claim 1 wherein the signal triggering apparatus comprises at least one device selected from the group consisting of:
microcontrollers, microprocessors, chips, timers, clocks, semiconductors, sensors, temperature sensors, galvanometers, flow detectors, air detectors, read-only memory (ROM) devices, programmable read-only memory (PROM) devices, field programmable read-only memory (FPROM) devices and one-time programmable non-volatile memory (OTP NVM) devices.

4. A system according to claim 1 further in combination with a catheter or medical device that is insertable or positionable within the body of a subject.

5. A system according to claim 4 wherein the catheter or medical device comprises a catheter, conduit, probe or other device useable for at least one purpose selected from: infusion or administration of a substance; withdrawal or drainage of a fluid or substance; monitoring or measurement of pressure; monitoring or measurement of arterial pressure; monitoring or measurement of venous pressure; monitoring or measurement of intracranial pressure; monitoring or measurement of any physiological variable; delivery of electrical impulses or stimuli and delivery or performance of a therapy.

6. A system according to claim 4 wherein the catheter or medical device is selected from the group consisting of: intravenous catheters; intra-arterial catheters; intracranial catheters; urinary catheters; Foley catheters; infusion catheters; sensing catheters; ostomy tubes; needles; drainage tubes; biliary tubes; venous access devices; medical tubing; feeding tubes; nasogastric tubes; sheath introducers; luer-lock extensions; anchor pads and other catheters, conduits and medical devices that are insertable into a subject's body.

7. A system according to claim 4 wherein the catheter or other medical device is for intravenous, intra-arterial, intravesicular, intracranial, or other insertion into the body of the subject.

8. A system according to claim 1 wherein the signal triggering apparatus causes the signaling apparatus to emit a signal upon expiration or impending expiration of a predetermined time period and comprises:
a switch apparatus for starting the time period and a timer.

9. A system according to a claim 1 wherein the signal triggering apparatus causes the signaling apparatus to emit a signal upon sensing of a change in a physiological or device-associated variable and comprises:
a sensor for sensing the variable.

10. A system according to claim 9 wherein the sensor senses when the variable has exceeded a predetermined or calculated limit and causes the signal emitting apparatus to emit at least one signal to notify a user that the variable has exceeded or is near exceeding the predetermined or calculated limit.

11. A system according to claim 10 wherein the sensor comprises a temperature sensor and the variable is temperature.

12. A system according to claim 10 wherein the sensor comprises a galvanic response sensor and the variable is galvanic skin response.

13. A system according to claim 10 wherein the sensor comprises a piezoelectric sensor and the variable is pulse rate.

14. A system according to claim 10 wherein the catheter or medical device comprises a lumen through which a fluid flows, wherein the sensor comprises a flow sensor and wherein the variable is too much or too little flow through the lumen.

15. A system according to claim 10 wherein the catheter or medical device comprises a lumen through which a fluid flows, wherein the sensor comprises an air detector and wherein the variable is the presence of air or air bubbles in the lumen.

16. A system according to claim 4 wherein the signaling system is positioned on or in the catheter, patch or medical device.

17. A system according to claim 4 wherein the signaling system is positioned on or in a secondary apparatus that is attached to or associated with the catheter, patch or medical device.

18. A system according to claim 17 wherein the secondary apparatus comprises a dressing, solution administration tube, connector, securement device, stabilization apparatus or securement apparatus.

19. A system according to claim 1 further comprising a switch for energizing circuitry of the system.

20. A system according to claim 19 wherein the switch comprises a twist tab which, when twisted, causes contacts to meet thereby completing an electrical circuit.

21. A system according to claim 1 wherein the signal triggering apparatus causes the signaling apparatus to emit a signal upon expiration of a time period selected from a plurality of possible time periods.

22. A system according to claim 21 further comprising a plurality of time period actuating switches, each of which is useable to actuate timing of a different time period.

23. A system according to claim 22 wherein the plurality of time period actuating switches comprise tabs which, when pressed together, will cause contact of electrical contacts to commence timing of a particular time period.

24. A system according to claim 1 wherein the components of the system are in or on a structure having an adhesive surface for adhering the structure to the body of a subject.

25. A method comprising the steps of:
positioning on or in a subject's body, or connecting to the subject's body, a catheter, tube, transdermal substance delivery device or other medical device that incorporates or is associated with a signaling system, said signaling system comprising a) signaling apparatus and b) a signal triggering apparatus which is programmed to cause the signaling apparatus to emit a signal upon occurrence of at least one event selected from a) expiration or impending expiration of a predetermined time period and b) a change in a physiological or device-associated variable; and
using the signaling system to generate a signal indicating at least one event selected from a) expiration or impending expiration of a predetermined time period and b) a change in a physiological or device-associated variable.

26. A method according to claim 25 wherein the signaling system comprises a microcontroller programmed to generate a signal upon expiration or impending expiration of a time for removal, replacement or replenishment of the medical device.

27. A method according to claim 25 wherein the signaling system comprises a sensor and a microcontroller programmed to generate a signal when the sensor senses a change in a physiological variable indicative of an infection or inflammation.

28. A method according to claim 25 wherein the signaling system comprises a sensor and a microcontroller that is programmed to a) generate a signal upon expiration or impending expiration of a time for removal, replacement or replenishment of the medical device and b) generate a signal when the sensor senses a change in a physiological variable indicative of an infection or inflammation.

29. A method according to claim 25 wherein the medical device comprises a catheter and wherein the signaling system is incorporated in or attached to at least one of:
the catheter; butterfly wings or tabs that are connected to the catheter; a tube that connects to the catheter; a connector that connects to the catheter; a connector or fitting in tubing that connects to the catheter; a securement device that is used to secure the catheter; a stabilization device that is used to stabilize the catheter; a dressing that is applied over an exteriorized portion of the catheter; a bracelet worn by the subject and an adherent member that is adhered to the subject's skin.

30. A method according to claim 25 wherein the medical device comprises a catheter inserted in the subject's body and wherein the signaling system is incorporated in or attached to a connector that connects to the catheter or to tubing that runs to the catheter.

31. A method according to claim 25 wherein the medical device comprises a catheter inserted in the subject's body and wherein the signaling system is incorporated in or attached to tubing that leads to the catheter.

32. A method according to claim 25 wherein the medical device comprises a catheter and wherein the signaling system is incorporated in or attached to a securement or stabilization device used to secure or stabilize the catheter.

33. A method according to claim 25 wherein the medical device comprises a transcutaneous substance delivery patch.

34. A method according to claim 33 wherein the signaling system comprises a microcontroller programmed to generate a signal upon expiration or impending expiration of a time for removal, replacement or replenishment of the transcutaneous substance delivery patch.

35. A method according to claim 34 wherein the signaling system further comprises a sensor for sensing a variable that affects the absorption or efficacy of the substance delivered by the transcutaneous substance delivery patch.

36. A method according to claim 34 wherein the physiological variable is temperature of the body under the patch or the patch itself.

37. A system for alerting a subject or caregiver, said system comprising:
a bracelet or other article worn by the subject or attached to the subject's body; and
a signaling system located in or on the bracelet or other article, said signaling system comprising (a) a signaling apparatus and (b) a signal triggering apparatus which causes the signaling apparatus to emit a signal upon occurrence of at least one event selected from: a) expiration or impending expiration of a predetermined time period and b) a change in a physiological or device-associated variable.

38. A transcutaneous substance delivery system comprising:
a transcutaneous substance delivery device positionable in contact with a subject's body and operative to deliver a substance to the subject;
a signaling system that emits a signal in response to at least one of:
a) time; and
b) change in a physiological or device-associated variable.

39. A system according to claim 38 wherein the transcutaneous substance delivery device comprises a transdermal substance delivery patch.

40. A system according to claim 39 wherein the transdermal substance delivery patch is selected from the group consisting of: single-layer drug-in-adhesive patches, multi-layer drug-in-adhesive patches, reservoir patches, matrix patches and vapor patches.

41. A system according to claim 38 wherein the signal emitting system comprises a timer and a signaling apparatus.

42. A system according to claim 38 wherein the signaling system includes a switch apparatus useable to energize circuitry of the signaling system.

43. A system according to claim 38 wherein the timer is operative to time a period during which the transcutaneous substance delivery device is intended to remain in contact with the subject's body and causes the signaling device to emit at least one signal to notify a user of the expiration or impending expiration of that period.

44. A system according to claim 38 wherein the signal emitting system comprises a sensor for sensing a physiological or device-related variable and a signaling apparatus.

45. A system according to claim 44 wherein the sensor senses when the variable has exceeded a predetermined or calculated limit and causes the signaling apparatus to emit at least one signal to notify a user that the variable has exceeded or is near exceeding the predetermined or calculated limit.

46. A system according to claim 44 wherein the sensor comprises a temperature sensor and the variable is temperature.

47. A system according to claim 46 wherein the temperature sensor senses temperature under the patch.

48. A system according to claim 44 wherein the sensor comprises a galvanic response sensor and the variable is galvanic skin response.

49. A system according to claim 44 wherein the sensor comprises a moisture sensor and the variable is moisture beneath or within the transcutaneous substance delivery device.

50. A system according to claim 44 wherein the sensor comprises a piezoelectric sensor and the variable is pulse rate.

51. A system according to claim 44 wherein the sensor comprises a photodetector and the variable is light exposure.
